SEQUENCE LISTING (3 sequences)

SEQ ID NO: 1 - DNA, 25 bp, Homo sapiens
tgcagctctcattttccatacagtc

SEQ ID NO: 2 - DNA, 23 bp, Homo sapiens
cctgttagagctactgcaattat

SEQ ID NO: 3 - DNA, 21 bp, Homo sapiens
ttcattacacctgcagctctc

(19) United States
(12) Patent Application Publication
(10) Pub. No.: US 2019/0358268 A1
(43) Pub. Date: Nov. 28, 2019

(54) Methods of Treating HIV Infection with Allogeneic CCR5 Null Umbilical Cord Blood Cells

(71) Applicant: Abraham J and Phyllis Katz Cord Blood Foundation, Cleveland, OH (US)

(72) Inventor: Mary LAUGHLIN, Cleveland, OH (US)

(21) Appl. No.: 16/484,180

(22) PCT Filed: Feb. 12, 2018
(86) PCT No.: PCT/US2018/017775

§ 371 Date: Aug. 7, 2019

Related U.S. Application Data
(60) Provisional application No. 62/457,392, filed on Feb. 10, 2017.

(51) Int. Cl. A61K 35/28; A61K 35/17; A61P 31/18

(52) U.S. Cl. CPC A61K 35/28; A61P 31/18; A61K 35/17

(57) ABSTRACT

Methods for providing allogeneic, immune-tolerant and virus-resistant umbilical cord blood cells are provided. Compositions and methods for treating a disease or condition in a subject by transplantation of allogeneic, immune-tolerant, and virus-resistant umbilical cord blood cells are provided. PCR-based methods for identifying donor cord blood having a CCR5 Δ32 genotype conferring HIV-resistance are provided.

### CROSS REFERENCES TO RELATED APPLICATIONS

This application claims priority benefit of U.S. Provisional Application No. 62/457,392, filed Feb. 10, 2017. The entire contents of which are incorporated by reference herein.

### SEQUENCE LISTING

The instant application contains a Sequence Listing which has been filed electronically in ASCII format and is hereby incorporated by reference in its entirety. Said ASCII copy, created on Feb. 9, 2018, is named 28478-0014_SL.txt and is 755 bytes in size.

### FIELD OF THE INVENTION

The present disclosure relates generally to methods for providing allogeneic, immune-tolerant, and virus-resistant umbilical cord blood cells, and methods for treating a disease or condition in a patient by transplantation of allogeneic, immune-tolerant, and virus-resistant umbilical cord blood cells.

### BACKGROUND

Human immunodeficiency virus (HIV) infection afflicts millions of people worldwide. Antiretroviral therapy (ART) provided a major breakthrough in managing HIV infection. Antiretroviral therapy extends and improves the quality of life in HIV infected patients by effectively suppressing, but not curing, HIV infection.

While antiretroviral therapy has proven successful, antiretroviral therapy is not sufficient to remove HIV from the body, and viral rebound is commonly observed after discontinuation of antiretroviral medication. Strict adherence to antiretroviral therapy is key to sustained HIV suppression, reduced risk of drug resistance, improved overall health, quality of life, and survival, as well as decreased risk of HIV transmission, and requires nearly all HIV-infected individuals to follow daily drug regimens for the entirety of their lives. Poor adherence is the major cause of therapeutic failure, and patient compliance with prescribed regimens is often comprised as a result of various behavioral, structural, and psychosocial barriers (e.g. mental illness, lack of social support, substance abuse, poverty, etc.) and other factors (e.g. regimen complexity). Antiretroviral medication is also toxic, and managing both short-term and long-term toxicities is critical to ensuring patient adherence and avoiding long-term adverse effects such as bone or renal toxicity, dyslipidemia, insulin resistance, or accelerated cardiovascular disease. Due to antiretroviral therapy's shortcomings, alternative therapeutic strategies are being explored to establish a cure for HIV.

One potential strategy for eradicating HIV is blood stem cell transplantation (BSCT). Blood stem cell transplantation involves the intravenous infusion of autologous or allogeneic blood stem cells, typically following a myeloablative or nonmyeloablative conditioning procedure, to reestablish hematopoietic function in patients whose bone marrow or immune system is damaged or defective, and has been used to treat a variety of blood diseases, autoimmune conditions, and malignant diseases. Myeloablative procedures involve conditioning a subject with high dose chemotherapy and radiotherapy to eradicate residual disease and recipient (host) immunity in preparation for healthy donor-derived blood stem cells (graft). The graft not only provides bone marrow reconstitution, but has an added benefit when the donor-derived lymphocytes mount a specific immune response to eradicate residual disease (graft versus host-disease effect). Nonmyeloablative procedures involve conditioning a subject with low dose chemotherapy and radiotherapy that is immunosuppressive but not myeloablative, and relies on the graft versus host-disease effect to eradicate damaged or defective cells.

Previous attempts to eradicate HIV by blood stem cell transplantation have not proven successful. In the absence of ART, transplanted stem cells are rapidly infected with endogenous HIV immediately after transplantation. Even with ART as a standard procedure to suppress viral burden, blood stem cell transplantation has failed to show any impact in controlling viral replication or eliminating the HIV viral reservoir. Moreover, in cases where ART was discontinued, the virus rebounds within a few days or weeks even in patients with completely undetectable viremia.

A promising modification to the BSCT approach is to confer HIV-resistance by transplanting virus-resistant blood stem cells. In the famous Berlin patient case, human leukocyte antigen (HLA) matched peripheral blood stem cells containing a homozygous 32-bp deletion in the chemokine receptor 5 gene CCR5 (CCR5-Δ32/Δ32) from an unrelated adult donor were transplanted into an AIDS patient with acute myeloid leukemia. Homozygous carriers of the 32-bp deletion in the CCR5 gene are highly resistant to infection by the most common forms of HIV-1, owing to the role of CCR5 as a co-receptor for HIV entry in CCR5+ CD4+ T-cells. The Berlin patient has remained without any evidence of HIV infection for many years after discontinuation of antiretroviral drug therapy, and the consensus is that he has been cured.

In view of the Berlin patient case, a potential BSCT strategy for HIV/AIDS treatment is to generate a new immune system to control HIV infection while at the same time destroying the endogenous reservoir of virus, thereby curing the infection. Success is largely predicated upon the creation of a durable (HIV-resistant) immune system through transplantation of resistant blood stem cells. HIV-resistant blood stem cells can resist infection by any endogenous virus and, in the absence of a suitable reservoir, the original virus is eliminated.

While a promising strategy, identifying a HLA-matched adult CCR5-Δ32/Δ32 donor for a given patient is not readily feasible in part because the prevalence of the homozygous variant allele is only about 0.8%-1% of individuals of northern European descent and much less in other ethnic groups. Further, when an adult donor is used for a BSCT, a very close HLA match between donor and patient is required. Accordingly, finding an adult donor who has a very close HLA match to a patient in need of a transplant and who is also homozygous for the CCR5-Δ32 allele is extremely difficult and will only rarely be possible.

Therefore, what are needed are improved methods for providing allogeneic, immune-tolerant, and virus-resistant blood stem cells, and improved methods for treating immune-related diseases or conditions by transplantation of allogeneic, immune-tolerant, and virus-resistant blood stem cells. More generally needed are improved methods for providing allogeneic, immune-tolerant, and virus-resistant blood cells, and improved methods for treating immune-related diseases or conditions by transplantation of allogeneic, immune-tolerant, and virus-resistant blood cells.

### SUMMARY OF THE INVENTION

The disclosure provides compositions and methods for providing a HIV-positive subject in need an allogeneic cell therapy of HIV-resistant umbilical cord blood cells. In certain embodiments, the methods for providing a HIV-positive subject in need of allogeneic cell therapy HIV-resistant umbilical cord blood cells comprise: screening a plurality of umbilical cord blood units to identify subject-compatible umbilical cord blood units that exhibit HLA matching with the subject; screening the plurality of umbilical cord blood units to identify HIV-resistant umbilical cord blood units; and providing subject-compatible and HIV-resistant umbilical cord blood cells from at least one of the umbilical cord blood units identified as being both subject-compatible and HIV-resistant.

The disclosure also provides compositions and methods for treating HIV in a subject having a hematological disease or condition in need of allogeneic cell therapy. In certain embodiments, the methods for treating HIV in a subject having a hematological disease or condition in need of allogeneic cell therapy comprise administering to the subject a composition comprising a therapeutically effective dose of subject-compatible HIV-resistant umbilical cord blood cells. In certain embodiments, the methods comprise administering to the subject a composition comprising a therapeutically effective dose of subject-compatible HIV-resistant allogeneic blood cells.

The disclosure also provides compositions and methods for providing a HIV-positive subject in need of allogeneic stem cell therapy, HIV-resistant umbilical cord blood stem cells. In certain embodiments, the methods for providing a HIV-positive subject in need of allogeneic stem cell therapy HIV-resistant umbilical cord blood stem cells include: screening a plurality of umbilical cord blood units to identify subject-compatible umbilical cord blood units that exhibit HLA matching with the subject; screening the plurality of umbilical cord blood units to identify HIV-resistant umbilical cord blood units; and providing subject-compatible HIV-resistant umbilical cord blood stem cells from at least one of the umbilical cord blood units identified as being both subject-compatible and HIV-resistant.

The disclosure also provides compositions and methods for treating HIV in a subject having a hematological disease or condition in need of allogeneic stem cell therapy. In certain embodiments, the methods for treating HIV in a subject having a hematological disease or condition in need of allogeneic stem cell therapy include administering to the subject a composition comprising a therapeutically effective dose of subject-compatible HIV-resistant blood stem cells.

The disclosure also provides methods for treating HIV in a subject by allogeneic stem cell therapy. In certain embodiments, the methods include administering to the subject a composition comprising a therapeutically effective dose of subject-compatible HIV-resistant blood stem cells.

In embodiments, the invention provides methods to detect CCR5 Δ32 homozygous, heterozygous, and wild type expression in donor blood, including dried blood spots of donor blood maintained in a cord blood bank, for use in treating HLA-compatible HIV subjects. In embodiments, the methods comprise screening cord blood for use in treating HIV subjects comprising conducting nested PCR-based assay of the cord blood to detect wild-type, heterozygous, or homozygous CCR5 Δ32 genotypes. HLA-compatibility of the cord blood for use in a particular subject can be conducted before or after a determination of CCR5 Δ32 genotype in the cord blood.

### BRIEF DESCRIPTION OF THE DRAWINGS

FIG. 1 shows wild-type CCR5 and CCR5 Δ32. A 32 base pair deletion in wild-type CCR5 produces a non-functional protein known as CCR5 Δ32. CCR5 Δ32 homozygotes are resistant to HIV-1 and the heterozygotes have a slower disease progression.

FIG. 2 shows a schematic for screening umbilical cord blood for HIV-resistance via CCR5 Δ32 homozygous deletions using nested polymerase chain reaction (PCR).

FIG. 3 shows exemplary results of screening wild type cell lines, a CCR5 Δ32 heterozygous cell line, and an umbilical cord blood spot for CCR5 Δ32 deletions using nested polymerase chain reaction (PCR). Shown in the 2% agarose gel image furthest to the right are amplicons for the CCR5 gene after two rounds of PCR. Lane 1 shows the DNA Ladder. Lanes 2 and 3 are the bands for the wild type cell line controls. Lane 4 shows the band for cord blood DNA.

### DETAILED DESCRIPTION

### I. Overview

The present disclosure relates to methods of providing allogeneic umbilical cord blood cells having a naturally occurring or artificially edited genetic mutation that confers HIV-resistance, and compositions and methods for treating a subject infected with HIV comprising transplanting into the subject allogeneic HLA-compatible umbilical cord blood cells having a genetic mutation that confers HIV-resistance. In some aspects, the present disclosure provides methods of treating HIV infected adults and children having hematologic disorders requiring allogeneic transplantation (e.g. sickle cell disease, severe thalassemia, and hematologic malignancies) with compositions comprising HLA-compatible umbilical cord blood stem cells, umbilical cord blood cells, or stem cells, having a genetic mutation that confers HIV-resistance.

The HIV-resistant umbilical cord blood stem cells can, for example, have a genetic mutation that prevents HIV from entering blood cells. Engrafted donor allogeneic HIV-resistant umbilical cord blood stem cells can mediate immune clearance of HIV infected blood cells in a subject, including cells containing HIV reservoirs, which in turn can ameliorate or cure the HIV infection. In some aspects, the present disclosure provides umbilical cord blood stem cells homozygous for the CCR5 Δ32 allele as a suitable graft source for HIV infected patients with hematologic disorders requiring transplant therapy. In some aspects, the present disclosure provides conferring HIV-resistance by transplantation of allogeneic umbilical cord blood stem cells homozygous for the CCR5 Δ32 allele.

Umbilical cord blood is a preferred source of blood cells relative to other donor sources (e.g. bone marrow or peripheral blood), due to several advantages. Blood cells derived from umbilical cord blood can be collected at no risk to the donor, and have lower risks of viral infection transmission and graft-versus-host-disease upon transplantation. Blood cells derived from umbilical cord blood also have greater long-term storage accessibility with immediate availability in a worldwide network of public banks. Blood cells derived from umbilical cord blood also have lower immune reactivity and do not require complete HLA matching (e.g. a mismatch at 1 or 2 loci is well tolerated), and therefore allow for more permissive HLA compatibility. Blood cells derived from umbilical cord blood also have wider availability of diverse tissue types and genotypes. Blood stem cells derived from umbilical cord blood have a further advantage in that these cells are not subject to the social and political controversy related to embryonic stem cells. Lastly, blood cells derived from umbilical cord blood have generally been shown to be equivalent to standard adult-derived bone marrow and mobilized peripheral blood cells.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

It is understood that aspects and embodiments of the invention described herein include “consisting” and/or “consisting essentially of” aspects and embodiments.

As used herein, “patient” or “subject” means an animal subject to be treated, with human patients being preferred.

As used herein, “proliferation” or “expansion” refers to the ability of a cell or population of cells to increase in number.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

As used herein, “therapeutically effective” refers to an amount of cells that is sufficient to treat or ameliorate, or in some manner reduce the symptoms associated with a disease or condition. When used with reference to a method, the method is sufficiently effective to treat or ameliorate, or in some manner reduce the symptoms associated with a disease or condition. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

As used herein, the term “treatment” embraces at least an amelioration of the symptoms associated with a disease or condition in the patient, where amelioration is used in a broad sense to refer to at least a reduction in the magnitude of a parameter, e.g. a symptom associated with the condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

The term “stem cell” refers to cells that are non-terminally differentiated cells having the ability to divide and to give rise to more specialized cells. Stem cells can emanate from all germinal layers (ectoderm, mesoderm and endoderm). Typical sources of stem cells include embryos, bone marrow, peripheral blood, umbilical cord blood, and placental blood. Stem cells are at least multipotent, meaning that they are capable of generating more than one tissue in an organism. Stem cells can also be pluripotent, meaning that they can differentiate into most tissues in an organism. For example, pluripotent stem cells can give rise to cells of the skin, liver, blood, muscle, bone, and the like.

The term “blood stem cell” refers to a stem cell from blood. Blood stem cells include stem cells such as hematopoietic stem cells and mesenchymal stromal cells.

The term “blood cell” refers to a cell from blood. Blood cells include cells such as blood stem cells, erythrocytes, leukocytes (e.g. lymphocytes such as natural killer cells, T cells, B cells), and thrombocytes.

The term “matching” refers to the degree of similarity between the genetic makeup of a donor source and a transplant recipient's genetic makeup. When tissues from different persons exhibit matching, that means that the tissues are immunologically compatible with each other.

The term “umbilical cord blood” refers to a source of pluripotent and/or multipotent stem cells obtained from the blood of umbilical cords that are left over after birth. Examples of stem cells found in umbilical cord blood include, but are not limited to, mesenchymal stem cells, hematopoietic stem cells, and progenitor cells. Mesenchymal stem cells and progenitor cells can typically differentiate into nerve cells, marrow stromal cells, chondrocytes, osteoblasts, adipocytes, myocytes, tenocytes, and ligament cells. Hematopoietic stem cells can typically give rise to cells of the lymphoid, myeloid, and erythroid lineages. Umbilical cord blood includes blood obtained from a neonate or fetus.

Umbilical cord blood also includes blood obtained from the umbilical cord or placenta of newborns.

The term “umbilical cord blood unit” refers to a volume of umbilical cord blood that is collected from a single donor.

The term “umbilical cord tissue” generally refers to tissue from an umbilical cord such as umbilical vein sub-endothelium, umbilical cord blood, amnion, placenta, amniotic fluid, microvillus, and Wharton's jelly.

Throughout this disclosure the methods and compositions are described in reference to umbilical cord blood. It will be appreciated by those of ordinary skill that umbilical cord tissue can be substituted for umbilical cord blood in the present methods and compositions. Thus, the methods and compositions of the present disclosure equally apply to umbilical cord tissue as they do to umbilical cord blood.

Throughout this disclosure some methods and compositions are described in reference to blood stem cells. It will be appreciated by those of ordinary skill that blood cells generally, not just blood stem cells, can be used in any of the present methods and compositions. Thus, the methods and compositions of the present disclosure equally apply to blood cells as they do to blood stem cells.

### II. Methods for Providing HIV-Resistant Umbilical Cord Blood Cells

Methods are described for providing a HIV-positive subject in need of allogeneic cell therapy with HIV-resistant umbilical cord blood cells. In embodiments, methods are also described for providing a HIV-positive subject in need of allogeneic cell therapy with allogeneic HLA-matched CCR5 Δ32/Δ32 umbilical cord blood cell grafts. In embodiments, the methods can include one or more of the following steps: screening a plurality of umbilical cord blood units to identify subject-compatible umbilical cord blood units that exhibit HLA matching with the subject; screening the plurality of subject-compatible umbilical cord blood units to identify HIV-resistant umbilical cord blood units; expanding and/or conditioning subject-compatible HIV-resistant umbilical cord blood cells from at least one of the umbilical cord blood units identified as being both subject-compatible and HIV-resistant; and/or providing the subject-compatible HIV-resistant umbilical cord blood cells from at least one of the umbilical cord blood units identified as being both subject-compatible and HIV-resistant.

Methods are provided for providing a HIV-positive subject in need of allogeneic stem cell therapy with HIV-resistant umbilical cord blood stem cells. Methods are also provided for providing a HIV-positive subject in need of allogeneic stem cell therapy with allogeneic partially HLA-matched CCR5 Δ32/Δ32 umbilical cord blood stem cell grafts. In embodiments, the methods can include one or more of the following steps: screening a plurality of umbilical cord blood units to identify subject-compatible umbilical cord blood units that exhibit partial HLA matching with the subject; screening the plurality of subject-compatible umbilical cord blood units to identify HIV-resistant umbilical cord blood units; expanding and/or conditioning subject-compatible HIV-resistant umbilical cord blood stem cells from at least one of the umbilical cord blood units identified as being both subject-compatible and HIV-resistant; and/or providing the subject-compatible HIV-resistant umbilical cord blood stem cells from at least one of the umbilical cord blood units identified as being both subject-compatible and HIV-resistant.

In embodiments, a subject is identified as requiring allogeneic blood cell transplantation and/or allogeneic blood stem cell transplantation. A subject can require allogeneic blood cell and/or allogeneic blood stem cell transplantation to treat HIV. A subject can require allogeneic blood cell and/or allogeneic blood stem cell transplantation to treat a hematological disease or condition such as blood diseases, autoimmune conditions, and malignant diseases. Exemplary hematological diseases or conditions include acute myeloid leukemia, acute lymphoblastic leukemia, chronic myeloid leukemia, chronic lymphocytic leukemia, myeloproliferative disorder, myelodysplastic syndrome, multiple myeloma, non-Hodgkin's lymphoma, Hodgkin's disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn error of metabolism, epidermolysis bullosa, severe congenital neutropenia, Shwachman-Diamond syndrome, Diamond-Blackfan anemia, and leukocyte adhesion deficiency. A subject can require allogeneic blood cell and/or allogeneic blood stem cell transplantation to co-treat HIV and a hematological disease or condition.

Blood cells suitable for use in the present disclosure can include blood stem cells such as hematopoietic stem cells and mesenchymal stromal cells. Blood cells suitable for use in the present disclosure can include differentiated cells (e.g. non-stem cells) such as leukocytes. Exemplary leukocytes suitable for use in the present disclosure can include lymphocytes such as natural killer cells, T cells, and B cells.

In embodiments, when a subject infected with HIV is identified as needing allogeneic cell therapy, the methods can include screening umbilical cord blood units, typically stored in umbilical cord blood banks, to identify donor umbilical cord blood units that are immunologically compatible with the subject. In embodiments, umbilical cord blood units can be partially matched against the subject using standard clinical HLA matching procedures. The degree of matching acceptable for umbilical cord blood is typically 4/6 loci or greater, with the loci being selected from HLA-A, HLA-B, and HLA-DRB1. HLA-A and HLA-B can be typed by means of the standard 2-stage complement-dependent microcytotoxicity assay or molecular typing, and antigens assigned as defined by the World Health Organization (WHO) HLA nomenclature committee. HLA-DRB1 type can be determined by hybridization of polymerase chain reaction (PCR)-amplified DNA with sequence-specific oligonucleotide probes (SSOPs), with sequencing if needed. In embodiments, the screening of umbilical cord blood units to identify subject-compatible umbilical cord blood units can include searching for suitable (e.g. HLA-matched) allogeneic umbilical cord blood graft donors via the National Marrow Donor Program (NMDP) and the World Marrow Donor Association (WMDA) registry databases. In embodiments, the screening of umbilical cord blood units to identify subject-compatible umbilical cord blood units can include comparing the similarity between the genetic makeup of a donor source and a transplant recipient's genetic makeup. In embodiments, the National Marrow Donor Program and the World Marrow Donor Association databases are searched to identify umbilical cord blood units that are immunologically compatible with a subject.

In embodiments, the methods can include screening umbilical cord blood units to identify which of the umbilical cord blood units are HIV-resistant. There are many naturally occurring mutations that confer HIV-resistance which are known to those of ordinary skill in the art, and methods of screening for such mutations are readily appreciated by those of ordinary skill. In one illustrative example, umbilical cord blood units can be screened/tested to determine whether the umbilical cord blood units are heterozygous or homozygous carriers of a gene which confers HIV-resistance, such as the 32-bp deletion in the CCR5 gene (e.g. CCR5 Δ32). The screening process can involve CCR5 genotypic analysis using a nested PCR-based assay system on DNA preparations extracted from cord blood spots, such as described herein.

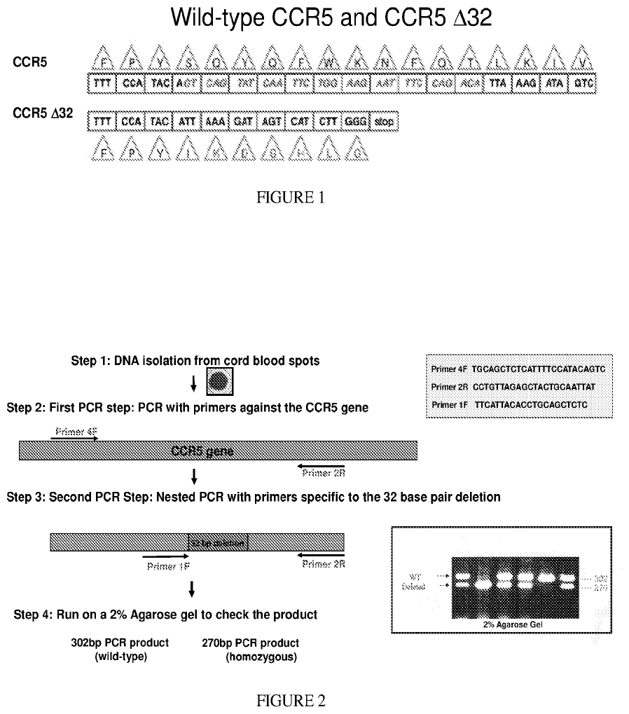

The immunocompatibility and HIV-resistance screening of umbilical cord blood units can be performed sequentially or in parallel. In some embodiments, the immunocompatibility screening of umbilical cord blood units is performed first, and the HIV-resistance screening step is only performed on immunocompatible umbilical cord blood units. In some embodiments, the HIV-resistance screening of umbilical cord blood units is performed first, and the immunocompatibility screening step is only performed on HIV-resistant umbilical cord blood units. In one embodiment, donor umbilical cord blood units are matched against a subject using standard clinical HLA matching, and either before or afterwards the partially matched umbilical cord blood units are screened to determine whether they are homozygous carriers of the 32-bp deletion in the CCR5 gene (e.g. CCR5 Δ32/Δ32), shown in FIG. 1.

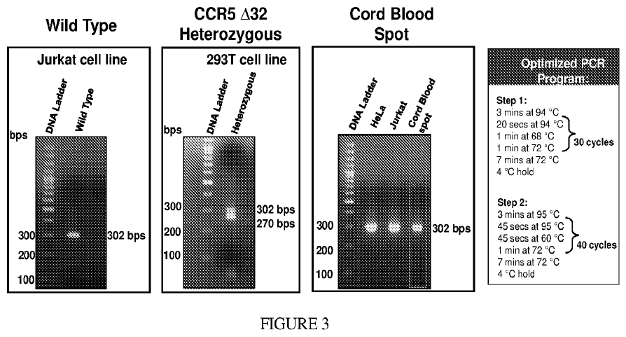

As further exemplified in FIGS. 2 and 3, the invention provides methods for determining CCR5 Δ32 homozygous, heterozygous, or wild type expression in donor blood, including dried blood spots of donor blood maintained in a cord blood bank, for use in treating HLA-compatible HIV patients. HLA-compatibility of the cord blood for use in a particular subject can be conducted before or after a determination of CCR5 Δ32 genotype in the cord blood. Therefore, CCR5 Δ32 genotype can be detected in a plurality of cord blood units in advance of identifying a HIV patient with HLA-compatibility for using the cord blood in a method of treatment.

In embodiments, the methods comprise identifying cord blood for use in treating HIV patients comprising conducting nested PCR-based assay of the cord blood to detect wild-type, heterozygous, or homozygous CCR5 Δ32 genotypes. In embodiments, detection of the genotype by the nested PCR-based assay is conducted using three unique PCR primers: a first primer pair specific for the CCR5 gene on either side of a 32 bp deletion causing HIV-resistance, which pair under PCR conditions to construct a first CCR5 amplicon, and a third primer specific for a CCR5 sequence between the first primer pair, and in embodiments is specific for a region adjacent to the 32 bp deletion site, wherein the third primer pairs with one of the primers of the first primer pair under PCR conditions to construct a second nested CCR5 amplicon. In embodiments, detection of the genotype by the nested PCR-based assay is conducted with the three primers comprising, or consisting essentially of, the nucleotides identified in SEQ ID No:1, SEQ ID No:2 and SEQ ID No:3, respectively. Determination of the genotype is made by evaluating whether the 32 bp deletion is present in the second amplicon, such as determined by size or weight relative to the first amplicon and/or additional control amplicons, e.g. in a gel electrophoresis.

In some embodiments, HLA-compatible umbilical cord blood cells can be made HIV-resistant via genetic mutations that are artificially created using well-known gene editing techniques, such as with the CRISPR-Cas9 system, by the deletion of the identified 32-bp sequence within one or both CCR5 alleles. The gene-edited HLA-compatible umbilical cord blood cells can be isolated and expanded prior to administration to the subject.

In some embodiments, certain immunocompatible HIV-resistant blood cells from at least one of the umbilical cord blood units identified as being both subject-compatible and HIV-resistant are separated/isolated from other umbilical cord blood cells in the umbilical cord blood unit. In some embodiments, certain blood cells are substantially separated from other cells in the umbilical cord blood unit to form a purified immunocompatible HIV-resistant blood cell composition. Methods for separating/purifying blood cells from umbilical cord blood are well-known in the art. In some embodiments, at least 75%, 85%, 86%, 87%, 88%, 89%, 90%, 91%, 92%, 93%, 94%, 95%, 96%, 97%, 98%, 99%, or more of the cells of the resulting composition are immunocompatible HIV-resistant blood cells. In some embodiments, the purity of immunocompatible HIV-resistant blood cells is equal to or greater than 75%, 85%, 86%, 87%, 88%, 89%, 90%, 91%, 92%, 93%, 94%, 95%, 96%, 97%, 98%, 99%, or more.

In some embodiments, immunocompatible HIV-resistant blood stem cells from at least one of the umbilical cord blood units identified as being both subject-compatible and HIV-resistant are separated/isolated from the umbilical cord blood in the umbilical cord blood unit. In some embodiments, immunocompatible HIV-resistant blood stem cells are substantially separated from other cells in the umbilical cord blood unit to form a purified immunocompatible HIV-resistant blood stem cell composition. Methods for separating/purifying blood stem cells from umbilical cord blood are well known in the art. In some embodiments, at least 75%, 85%, 86%, 87%, 88%, 89%, 90%, 91%, 92%, 93%, 94%, 95%, 96%, 97%, 98%, 99%, or more of the cells of the resulting composition are immunocompatible HIV-resistant blood stem cells. In some embodiments, the purity of immunocompatible HIV-resistant blood stem cells is equal to or greater than 75%, 85%, 86%, 87%, 88%, 89%, 90%, 91%, 92%, 93%, 94%, 95%, 96%, 97%, 98%, 99%, or more.

In embodiments, immunocompatible HIV-resistant umbilical cord blood cells from at least one of the umbilical cord blood units identified as being both subject-compatible and HIV-resistant are conditioned and/or expanded ex vivo. In some embodiments, ex vivo expansion of blood cells from the umbilical cord blood units is one of the ways to increase umbilical cord blood cells for transplantation and to facilitate engraftment. In some embodiments, ex vivo conditioning of blood cells from the umbilical cord blood units is one of the ways to prepare umbilical cord blood cells for transplantation and to facilitate engraftment. Suitable methods for expanding blood cells from umbilical cord blood are known to those of ordinary skill. Suitable methods for conditioning blood cells from umbilical cord blood are known to those of ordinary skill.

In embodiments, immunocompatible HIV-resistant umbilical cord blood stem cells from at least one of the umbilical cord blood units identified as being both subject-compatible and HIV-resistant are conditioned and/or expanded ex vivo. In some embodiments, ex vivo expansion of blood stem cells from the umbilical cord blood units is one of the ways to increase umbilical cord blood stem cells for transplantation and to facilitate engraftment. In some embodiments, ex vivo conditioning of blood stem cells from the umbilical cord blood units is one of the ways to prepare umbilical cord blood stem cells for transplantation and to facilitate engraftment. Suitable methods for expanding blood stem cells from umbilical cord blood are known to those of ordinary skill. Suitable methods for conditioning blood stem cells from umbilical cord blood are known to those of ordinary skill.

In some embodiments, immunocompatible HIV-resistant umbilical cord blood cells are expanded at least 2-fold, at least 3-fold, 4, 5, 6, 7, 8, 9, 10, 50, 100, 200, 300, 500, or at least 800-fold. In some embodiments, compositions comprising the immunocompatible HIV-resistant umbilical cord blood cells contain a clinically relevant number or population of immunocompatible HIV-resistant umbilical cord blood cells. In some embodiments, compositions include about 10³, about 10⁴, about 10⁵ cells, about 10⁶ cells, about 10⁷ cells, about 10⁸ cells, about 10⁹ cells, about 10¹⁰ cells or more. In some embodiments, the number of cells present in the composition will depend upon the ultimate use for which the composition is intended, e.g., the disease or state or condition, patient condition (e.g., size, weight, health, etc.), and other health-related parameters that a skilled artisan would readily understand. In addition, in some embodiments, the clinically relevant number of cells can be apportioned into multiple infusions that cumulatively equal or exceed the desired administration, e.g., 10⁹ or 10¹⁰ cells.

In some embodiments, immunocompatible HIV-resistant umbilical cord blood stem cells are expanded at least 2-fold, at least 3-fold, 4, 5, 6, 7, 8, 9, 10, 50, 100, 200, 300, 500, or at least 800-fold. In some embodiments, compositions comprising the immunocompatible HIV-resistant umbilical cord blood stem cells contain a clinically relevant number or population of immunocompatible HIV-resistant umbilical cord blood stem cells. In some embodiments, compositions include about 10³, about 10⁴, about 10⁵ cells, about 10⁶ cells, about 10⁷ cells, about 10⁸ cells, about 10⁹ cells, about 10¹⁰ cells or more. In some embodiments, the number of cells present in the composition will depend upon the ultimate use for which the composition is intended, e.g., the disease or state or condition, patient condition (e.g., size, weight, health, etc.), and other health-related parameters that a skilled artisan would readily understand. In addition, in some embodiments, the clinically relevant number of cells can be apportioned into multiple infusions that cumulatively equal or exceed the desired administration, e.g., 10⁹ or 10¹⁰ cells.

In embodiments, the immunocompatible HIV-resistant umbilical cord blood cells and/or the immunocompatible HIV-resistant umbilical cord blood stem cells from at least one of the umbilical cord blood units identified as being both subject-compatible and HIV-resistant are provided. The immunocompatible HIV-resistant umbilical cord blood cells and/or the immunocompatible HIV-resistant umbilical cord blood stem cells can be provided to any suitable person or entity such as, for example, a patient, a clinician treating the patient, or a cell bank. The immunocompatible HIV-resistant umbilical cord blood cells and/or the immunocompatible HIV-resistant umbilical cord blood stem cells can be provided in a form for immediate use or they can be provided in a stored form for later use (e.g. frozen).

In embodiments, umbilical cord blood can originate from a variety of animal sources including, for example, humans.

In embodiments, compositions comprising immunocompatible HIV-resistant umbilical cord blood cells and/or immunocompatible HIV-resistant umbilical cord blood stem cells are provided. In embodiments, the compositions and/or cells of the composition are non-naturally occurring. In embodiments, the compositions and/or cells of the composition are not naturally occurring because cells of the composition are the result of one or more of isolation, purification, expansion, partial differentiation, ex vivo conditioning, gene editing, and the like.

In embodiments, compositions comprising a culture of substantially purified HIV-resistant umbilical cord blood-derived cells are provided. In embodiments, compositions comprising a culture of substantially purified CCR5 Δ32/Δ32 homozygous umbilical cord blood-derived cells are provided.

In embodiments, compositions comprising a culture of substantially purified HIV-resistant umbilical cord blood-derived stem cells are provided. In embodiments, compositions comprising a culture of substantially purified CCR5 Δ32/Δ32 homozygous umbilical cord blood-derived stem cells are provided.

In embodiments, a cell composition comprising a culture of substantially purified HIV-resistant umbilical cord blood-derived cells is provided. In embodiments, the culture is a culture of substantially purified CCR5 Δ32 homozygous umbilical cord blood-derived cells. In embodiments, the cells are human blood cells. In embodiments, the cells comprise T cells. In embodiments, the cells comprise natural killer cells. In embodiments, the cells comprise blood stem cells.

In embodiments, a stem cell composition comprising a culture of substantially purified HIV-resistant umbilical cord blood-derived stem cells is provided. In embodiments, the culture is a culture of substantially purified CCR5 Δ32 homozygous umbilical cord blood-derived stem cells. In embodiments, the stem cells are human blood stem cells. In embodiments, the stem cells comprise hematopoietic stem cells. In embodiments, the stem cells comprise mesenchymal stromal cells.

In embodiments, a therapeutic composition comprising a therapeutically effective dose of substantially purified HIV-resistant umbilical cord blood-derived cells is provided. In embodiments, the therapeutically effective dose is a therapeutically effective dose of substantially purified CCR5 Δ32 homozygous umbilical cord blood-derived cells.

In embodiments, the cells are human blood cells. In embodiments, the cells comprise T cells. In embodiments, the cells comprise natural killer cells. In embodiments, the cells comprise blood stem cells. In embodiments, the stem cells comprise hematopoietic stem cells. In embodiments, the stem cells comprise mesenchymal stromal cells.

### III. Exemplary Uses of HIV-Resistant Umbilical Cord Blood Cells

Compositions and methods are provided for treating HIV in a subject having a hematological disease or condition in need of allogeneic cell therapy. In embodiments, the methods include: administering to the subject a composition comprising a therapeutically effective dose of subject-compatible HIV-resistant umbilical cord blood cells provided in accordance with the methods of this disclosure. Methods are also provided for treating HIV in a subject by allogeneic cell therapy. In embodiments, the methods include administering to the subject a composition comprising a therapeutically effective dose of subject-compatible HIV-resistant umbilical cord blood cells provided in accordance with the methods of this disclosure.

Methods are provided for treating HIV in a subject having a hematological disease or condition that needs allogeneic stem cell therapy. In embodiments, the methods include: administering to the subject a composition comprising a therapeutically effective dose of subject-compatible HIV-resistant umbilical cord blood stem cells provided in accordance with the methods of this disclosure. Methods are also provided for treating HIV in a subject by allogeneic stem cell therapy. In embodiments, the methods include administering to the subject a composition comprising a therapeutically effective dose of subject-compatible HIV-resistant umbilical cord blood stem cells provided in accordance with the methods of this disclosure.

In embodiments, a subject can require allogeneic blood cell and/or allogeneic blood stem cell transplantation to treat HIV and/or a hematological disease or condition. In embodiments, the subject has a hematological disease or condition such as blood diseases, autoimmune conditions, and malignant diseases. Exemplary hematological diseases or conditions include acute myeloid leukemia, acute lymphoblastic leukemia, chronic myeloid leukemia, chronic lymphocytic leukemia, myeloproliferative disorder, myelodysplastic syndrome, multiple myeloma, non-Hodgkin's lymphoma, Hodgkin's disease, aplastic anemia, pure red-cell aplasia, paroxysmal nocturnal hemoglobinuria, fanconi anemia, thalassemia major, sickle cell anemia, severe combined immunodeficiency (SCID), Wiskott-Aldrich syndrome, hemophagocytic lymphohistiocytosis, inborn error of metabolism, epidermolysis bullosa, severe congenital neutropenia, Shwachman-Diamond syndrome, Diamond-Blackfan anemia, and leukocyte adhesion deficiency.

In embodiments, a therapeutically effective amount of subject-compatible HIV-resistant umbilical cord blood cells and/or umbilical cord blood stem cells can be administered to a subject with a pharmaceutically acceptable carrier. Administration routes may include any suitable means, including, but not limited to, intravascularly (intravenously or intra-arterially). In some embodiments, a preferred administration route is by intravenous (IV) infusion. In some embodiments, the particular mode of administration selected will depend upon the particular treatment, disease state or condition of the patient, the nature or administration route of other drugs or therapeutics administered to the subject.

In embodiments, about 10⁵-10¹¹ cells can be administered in a volume of a 5 ml to 1 liter, 50 ml to 250 ml, 50 ml to 150, and typically 100 ml. In some embodiments, the volume will depend upon the disorder treated, the route of administration, the patient's condition, disease state, etc. The cells can be administered in a single dose or in several doses over selected time intervals, for example to titrate the dose.

In some embodiments, the methods of treatment include administering to a subject in need thereof a therapeutically effective amount of a composition comprising allogeneic partially HLA-matched CCR5 Δ32/Δ32 umbilical cord blood cells and/or umbilical cord blood stem cells to disrupt CCR5 functionality. In some embodiments, administration of a therapeutically effective dose of subject-compatible HIV-resistant umbilical cord blood cells and/or umbilical cord blood stem cells results in improvements by eliminating most or all of the HIV infected patient cells via immune clearance. In some embodiments, administration of HIV resistant CCR5 Δ32/Δ32 umbilical cord blood cells and/or umbilical cord blood stem cells can mediate immune clearance of HIV-infected recipient blood cells including the latent HIV reservoir in memory T lymphocytes and tissue macrophages.

In some embodiments, the methods of treatment include a myeloablative conditioning procedure (e.g. high dose chemotherapy and/or radiotherapy) prior to administration or transplantation of the umbilical cord blood cells and/or umbilical cord blood stem cells. In some embodiments, the methods of treatment include a nonmyeloablative conditioning procedure (e.g. low dose chemotherapy and/or radiotherapy) prior to administration or transplantation of the umbilical cord blood cells and/or umbilical cord blood stem cells.

In embodiments, the methods of treatment include first reducing the number of HIV-infected cells, including the HIV reservoir, in the recipient with chemotherapeutic drugs with or without total body irradiation (e.g. BuCy, FluBu, FluMel, FluTBI), and then eliminating any remaining reservoir by graft vs. host effect following umbilical cord blood cell and/or umbilical cord blood stem cell infusion.

### EXAMPLES

Identification of HIV-Resistance in Cells by CCR5 and its Null Phenotype, CCR5Δ32, from Umbilical Cord Blood Spots.

Equipment and Reagents
Umbilical Cord Blood spot
Sharps Container
Biohazard Waste Container
Protective Nitrile Gloves
Sterile Scissors
15 ml and 50 mL Falcon Tubes
5 ml, 10 ml and 25 ml Sterile Serological pipettes
Pipette tips
Sterile glass conical flask
Millipore water
Pipette Aid
1.5 ml Microcentrifuge tube
Heat Block, Fisher Scientific Isotemp
Eppendorf 5424 Microcentrifuge (15,000 rpm, 21,130×g, 120 V, 60 Hz, #4062-0401)
QIAamp DNA Micro Kit (Ref 56304; Lot 154038977)
Bio-Rad Certified PCR Agarose (Bio-Rad #161-3103)
Fisher Biotech Electrophoresis system Mini Horizontal unit (Fisher Scientific, FB-SB-710)
SubCell GT UV Transparent Mini Gel Tray (Bio-Rad#1704436)
Vortex Genie 2
10×TBE (Bio-Rad#161-0733)
Bio-Rad C1000 Touch Thermal Cycler
Bio-Rad Power Pac 1000
Microwave (Sanyo, Super Shower wave)
dNTP Set 100 mM solution (ThermoFisher Scientific, #R0181)
GeneRuler 100 bp Plus DNA Ladder, 0.1 μg/μl (Thermo Scientific # SM0323)
AmpliTaq Gold DNA Polymerase with Buffer I (ThermoFisher Scientific #N8080240)
Ethidium Bromide, 10 mg/ml (ThermoFisher Scientific #15585011)
5× Nucleic Acid Sample Loading Buffer (Bio-Rad #1610767)
UV Transilluminator, Spectroline Select Series (TI-312E)
PCR Primers (Integrated DNA Technologies);

(SEQ ID No: 1)
Primer 4F: 5′-TGCAGCTCTCATTTTCCATACAGTC-3′;
(SEQ ID No: 2)
Primer 2R: 5′-CCTGTTAGAGCTACTGCAATTAT-3′;
(SEQ ID No: 3)
Primer 1F: 5′-TTCATTACACCTGCAGCTCTC-3′

Procedure for the identification and screening of CCR5 and CCR5Δ32 from umbilical cord blood spots, using DNA isolation method followed by a nested PCR approach.
Obtain umbilical cord blood spots and store at 4° C. refrigerator if not used immediately.
Decontaminate and clean the work space with 70% Ethyl alcohol, before proceeding to the DNA isolation steps.
Carefully cut each cord blood spot into small pieces, using a sterile, autoclaved pair of scissors and put the pieces into a 1.5 ml microcentrifuge tube.
For the DNA isolation steps, use QIAamp DNA micro kit and follow the protocol mentioned in the QIAamp DNA micro handbook; Isolation of Genomic DNA from Dried Blood spots, Page 19.
Store the isolated DNA at −20° C., if not used immediately, otherwise proceed to the steps mentioned below.
Prepare a reaction mixture for the first PCR step; reaction mixture for a single PCR will contain 2 μl UCB DNA, 30 pmole Primer 4F, 30 pmole Primer 2R, 250 μM dNTP mix, 1 μl AmpliTaq Gold DNA Polymerase, 2.5 μl 10× buffer I and 17.25 μl water in a 25 μl total volume.
The first round PCR reaction was carried out in a Bio-Rad C1000 Touch Thermal Cycler for 3 min at 94° C. (1 cycle); 20 seconds at 94° C., 1 min at 68° C., 1 min at 72° C. (30 cycles); 7 min at 72° C. (1 cycle) and 4° C. hold forever.
Store the amplicon from the first PCR at −20° C., if not used immediately, otherwise proceed to the second round of PCR.
Perform a second round of PCR with the amplicon from the first PCR.
Prepare a reaction mixture for the second PCR step; reaction mixture for a single

PCR will contain 2 μl first round amplicon, 30 pmole Primer 1F, 30 pmole Primer 2R, 250 μM dNTP mix, 1 μl AmpliTaq Gold DNA Polymerase, 2.5 μl 10× buffer I and 17.25 μl water in a 25 μl volume.
The second round PCR reaction was carried out for 3 min at 95° C. (1 cycle); 45 seconds at 95° C., 45 seconds at 60° C., 45 seconds at 72° C. (40 cycles); 7 min at 72° C. (1 cycle) and 4° C. hold.
Add 5 μl 5× Nucleic Acid Sample Loading Buffer and store the second PCR amplicon at −20° C., if not used immediately, otherwise run in an agarose gel.
Heat 1 gm agarose (using #161-3103) in 50 ml 1×TBE buffer to prepare a 2% agarose gel solution. Add 5 μl EtBr solution (10 mg/ml) and pour the gel solution in a UV transparent mini gel tray. Let it to solidify for about 30 min-1 hr.
Load the PCR amplicons in the different lanes of the 2% agarose gel, along with a 100 bp ladder in the first lane.
Run the 2% agarose gel with 1×TBE buffer, using a Fisher Biotech Electrophoresis system Mini Horizontal unit at a constant voltage of 90 volts.
Track the tracking dye. Do not let the tracking dye run out of the gel.
Analyze the bands under a UV Transilluminator, every half an hour until a better resolution of the bands is achieved.

Results
DNA from umbilical cord blood spots can be isolated and amplicons for CCR5 wild Type (302 bps), Heterozygous (302 bps, 270 bps) or Homozygous (270 bps) can be identified by this method, as shown in FIG. 2.
Stem cells from the HIV-resistant CCR5 Δ32 homozygous sample can be isolated from the umbilical cord sample and utilized in the present methods, cryopreserved for future use, or cultured for expansion.

1. A method for providing a HIV-positive subject in need of allogeneic cell therapy HIV-resistant umbilical cord blood cells comprising:
screening a plurality of umbilical cord blood units to identify subject-compatible umbilical cord blood units that exhibit HLA matching with the subject;
screening the plurality of umbilical cord blood units to identify HIV-resistant umbilical cord blood units; and
providing subject-compatible HIV-resistant umbilical cord blood cells from at least one of the umbilical cord blood units identified as being both subject-compatible and HIV-resistant.

2. The method of claim 1, wherein the plurality of umbilical cord blood units are human umbilical cord blood units.

3. The method of claim 1, further comprising expanding the subject-compatible HIV-resistant umbilical cord blood cells ex vivo.

4. The method of claim 1, further comprising conditioning the subject-compatible HIV-resistant umbilical cord blood cells ex vivo.

5. The method of claim 1, wherein the subject-compatible HIV-resistant umbilical cord blood cells comprise hematopoietic stem cells.

6. The method of claim 1, wherein the subject-compatible HIV-resistant umbilical cord blood cells comprise mesenchymal stromal cells.

7. The method of claim 1, wherein the subject-compatible HIV-resistant umbilical cord blood cells comprise natural killer cells.

8. The method of claim 1, wherein the subject-compatible HIV-resistant umbilical cord blood cells comprise T cells.

9. The method of claim 1, wherein the step of screening to identify HIV-resistant umbilical cord blood units comprises screening the umbilical cord blood units for CCR5 Δ32 homozygous deletions using nested polymerase chain reaction (PCR) on umbilical cord dried blood spots.

10. The method of claim 1, wherein the subject-compatible HIV-resistant umbilical cord blood cells comprise CCR5 Δ32 homozygous cells.

11. A method for treating HIV in a subject having a hematological disease or condition that needs allogeneic cell therapy comprising:
administering to the subject a composition comprising a therapeutically effective dose of subject-compatible HIV-resistant umbilical cord blood cells provided according to claim 1.

12. The method of claim 11, wherein the hematological disease or condition is an acute myeloid leukemia, an acute lymphoblastic leukemia, a chronic myeloid leukemia, a chronic lymphocytic leukemia, a myeloproliferative disorder, a myelodysplastic syndrome, a multiple myeloma, a non-Hodgkin's lymphoma, a Hodgkin's disease, an aplastic anemia, a pure red-cell aplasia, a paroxysmal nocturnal hemoglobinuria, a Fanconi anemia, a thalassemia major, a sickle cell anemia, a severe combined immunodeficiency (SCID), a Wiskott-Aldrich syndrome, a hemophagocytic lymphohistiocytosis, an inborn error of metabolism, an epidermolysis bullosa, a severe congenital neutropenia, a Shwachman-Diamond syndrome, a Diamond-Blackfan anemia, a leukocyte adhesion deficiency, or a combination thereof.

13. The method of claim 11, wherein the dose of subject-compatible HIV-resistant umbilical cord blood cells are therapeutically effective to treat the hematological disease or condition.

14. The method of claim 11, wherein the dose of subject-compatible HIV-resistant umbilical cord blood cells are therapeutically effective to treat HIV.

15. A method for treating HIV in a subject by allogeneic cell therapy comprising:
administering to the subject a composition comprising a therapeutically effective dose of subject-compatible umbilical cord blood-derived CCR5 Δ32 homozygous cells.

16. A cell composition comprising:
a culture of substantially purified HIV-resistant umbilical cord blood-derived cells.

17. The composition of claim 16, wherein the culture is a culture of substantially purified CCR5 Δ32 homozygous umbilical cord blood-derived cells.

18. The composition of claim 16, wherein the cells are human cord blood cells.

19. The composition of claim 16, wherein the cells comprise hematopoietic stem cells.

20. The composition of claim 16, wherein the cells comprise mesenchymal stromal cells.

21. The composition of claim 16, wherein the cells comprise natural killer cells.

22. The composition of claim 16, wherein the cells comprise T cells.

23. A therapeutic composition comprising:
a therapeutically effective dose of substantially purified HIV-resistant umbilical cord blood-derived cells.

24. The composition of claim 23, wherein the therapeutically effective dose is a therapeutically effective dose of substantially purified CCR5 Δ32 homozygous umbilical cord blood-derived cells.

25. The composition of claim 24, wherein the cells are human blood cells.

26. The composition of claim 25, wherein the cells comprise hematopoietic stem cells.

27. The composition of claim 25, wherein the cells comprise mesenchymal stromal cells.

28. The composition of claim 25, wherein the cells comprise natural killer cells.

29. The composition of claim 25, wherein the cells comprise T cells.

30. A method of identifying cord blood for use in treating a HIV subject comprising conducting nested PCR-based assay on cord blood to detect wild-type, heterozygous, or homozygous CCR5 for a Δ32 genotype causing HIV-resistance, wherein detecting a heterozygous or homozygous CCR5 Δ32 genotype identifies cord blood for use in treating the HIV subject.

31. The method of claim 30, wherein detection of the genotype by the nested PCR-based assay is conducted with three unique PCR primers: a first primer pair specific for the CCR5 gene on either side of a 32 bp deletion causing HIV-resistance, which pair under PCR conditions to construct a first CCR5 amplicon, and a third primer specific for a sequence between the first primer pair, wherein the third primer pairs with one of the primers of the first primer pair under PCR conditions to construct a second nested CCR5 amplicon.

32. The method of claim 31, wherein the third primer specific for the sequence between the first primer pair is adjacent to the 32 bp deletion.

33. The method of claim 32, wherein detection of the genotype by the nested PCR-based assay is conducted with the three primers identified by SEQ ID No:1, SEQ ID No:2 and SEQ ID No:3, respectively.

34. The method of claim 30, wherein multiple units of cord blood are individually tested by dried blood spot analysis by the method to identify cord blood for use in treating HIV subjects.

35. The method of claim 34, wherein cord blood identified with a CCR5 Δ32 genotype is further tested for HLA-compatibility with an individual subject.
